(19) United States
(12) Patent Application Publication
(10) Pub. No.: US 2025/0376713 A1
(43) Pub. Date: Dec. 11, 2025

(54) METHOD FOR DNA OR RNA AMPLIFICATION FROM URINE, SALIVA AND/OR MOUTHWASH SAMPLES

(71) Applicant: Testmate Health SA, Vaud (CH)

(72) Inventor: Gaëtan Cherbuin, Chavannes-près-Renens (CH)

(21) Appl. No.: 18/877,991

(22) PCT Filed: Jun. 20, 2023
(86) PCT No.: PCT/EP2023/066691

§ 371 Date: Dec. 20, 2024

(30) Foreign Application Priority Data
Jun. 20, 2022 (EP) 22179963.8
Jun. 20, 2022 (EP) 22179966.1

(51) Int. Cl. C12Q 1/6806; C12Q 1/6844

(52) U.S. Cl. CPC C12Q 1/6806; C12Q 1/6844

(57) ABSTRACT

A method for sample preparation and/or DNA amplification from urine, saliva and/or mouthwash samples is disclosed. The method involves providing a sample preparation system comprising a filter and providing a urine, saliva and/or mouthwash sample, then filtering at least a portion of the sample through the filter, thereby retaining bacteria, fungi, viruses protists and/or cancerous or precancerous cells in the filter. Subsequently a lysis reagent is applied to the filter. Finally, the DNA found in the lysate is amplified.

The present invention relates to methods for sample preparation and/or DNA amplification from urine, saliva and/or mouthwash samples. The methods may be employed, e.g., for determining the presence of pathogens in the urine, such as bacteria. More specifically, the invention relates to a method for DNA amplification suitable in the screening of urine, saliva and/or mouthwash samples for sexually transmitted diseases (such as infections caused by bacteria—e.g. chlamydia or gonorrhea—or infections caused by protists-such as e.g. Trichomonas vaginalis), bacterial infections (such as urinary tract infection, urethritis, cystitis, pyelonephritis, gingivitis, and/or periodontitis), and/or protist infections. Alternatively or additionally, the method for DNA amplification may be suitable in the screening of urine, saliva and/or mouthwash samples for oncological conditions (e.g., cancerous or precancerous conditions, such as for example conditions to be determined based on the presence of cancerous or precancerous cells in the urine, saliva or mouthwash sample). The invention particularly relates to a method for amplifying DNA from samples that may be performed by a user at home or at a point of care.

Isolation of DNA from urine samples for the purpose of DNA amplification is traditionally achieved by taking a sample of whole urine (also referred to as “crude” urine in the art), e.g. an aliquot of 1 ml, extracting DNA from the sample by methods including column purification (in particular with silica beads), and performing the respective DNA amplification procedure (e.g., PCR or Real-Time PCR).

Isolation and amplification of DNA in this manner requires dedicated materials, instruments and experience. These methods may be time-intensive, complex, and/or costly. Furthermore, they are less suitable for home settings or at a point of care.

It is thus an object of the present invention to propose improved methods for sample preparation and/or DNA or RNA amplification from urine samples. It is a further object to propose improved methods for sample preparation and/or DNA or RNA amplification from saliva and/or mouthwash samples.

It is a further object to provide for a simple and economical preparation of urine, saliva and/or mouthwash samples for DNA or RNA amplification, in particular a preparation that may be performed in an at-home or point of care setting.

Another object of the present invention to provide a simple and cost-efficient method resulting in a DNA or RNA sample of sufficient quality to be used in isothermal DNA or RNA amplification methods, e.g. in Loop-Mediated Isothermal Amplification (LAMP), for example a LAMP that is performed in an at-home or point of care setting.

According to an aspect, the present invention relates to a method for sample preparation and/or DNA or RNA amplification from urine, saliva and/or mouthwash samples. The method comprises the steps of (i) providing a sample preparation system comprising a filter, (ii) providing a urine sample, a saliva sample and/or a mouthwash sample, (iii) filtering at least a portion of the urine sample through the filter, thereby retaining bacteria, fungi, viruses, protists and/or cancerous or precancerous cells in the filter, (iv) applying a lysis reagent to the filter, and (v) amplifying DNA or RNA in the lysate, preferably DNA or RNA of the bacteria, fungi, viruses, protists and/or cancerous or precancerous cells contained in the lysate. Preferably, these steps are performed in the stated order.

In other words, the method of the invention may comprise collecting cells, then lyse them (e.g. with an alkaline reagent). Subsequently, the lysis reagent may be neutralized by another reagent (which may also be referred to as a neutralization reagent or neutralization buffer herein). The combination of cells, lysis reagent and neutralization reagent may produce a crude lysate. The crude lysate (at least a fraction thereof) may then be used for a DNA or RNA amplification. The lysis reagent may comprise a lysis buffer.

Preferably, at least steps (ii) and (iii) are performed at home or at a point of care (e.g., a medical practice) and/or not a laboratory. More preferably, at least steps (ii), (iii), and (iv) are performed at home or at a point of care and/or not a laboratory. Even more preferably, at least steps (ii), (iii), (iv), and (v) are performed at home or at a point of care and/or not a laboratory. Steps (i) to (v) may all be performed at home or at a point of care and/or not a laboratory.

In other words, at least in an at-home setting, the urine sample preferably is micturated by the same individuum, patient and/or user that subsequently performs step (iii), preferably also step (iv), more preferably also step (v). Similarly, the saliva or mouthwash sample preferably is given by the same individuum, patient and/or user that subsequently performs step (iii), preferably also step (iv), more preferably also step (v).

The urine sample is preferably whole urine.

Preferably, the whole urine is micturated 1 hour or less, 30 minutes or less, 15 minutes or less, or 10 minutes or less before filtering at least the portion of the urine sample through the filter. Preferably, the saliva or mouthwash sample is given 1 hour or less, 30 minutes or less, 15 minutes or less, or 10 minutes or less before filtering at least the portion of the sample through the filter.

The method preferably comprises dispensing the filtered urine, saliva and/or mouthwash through an outlet of the sample preparation system, e.g. into a drain or toilet.

The step of filtering at least a portion of the sample through the filter may comprise filtering a volume of at least at least 4 ml, at least 5 ml, at least 6 ml, at least 8 ml at least 10 ml, at least 20 ml, or at least 50 ml of the sample through the filter. Volumes of at least 6 ml or 8 mL have been shown to be particularly suitable for achieving a reliable result in preliminary tests but it will be appreciated that lower volumes may be used, as long as a sufficient sensitivity is obtained.

Preferably, the filter has a pore size no greater than 1.0 μm, more preferably no greater than 0.8 μm, even more preferably no greater than 0.6 μm, most preferably no greater than 0.5 μm.

Preferably the filter has a pore size no greater than 0.45 μm, more preferably no greater than 0.4 μm, most preferably no greater than 0.22 μm. The bacteria filter may have a pore size of at least 0.10 μm, preferably at least 0.20 μm.

The whole urine, saliva or mouthwash may be pre-filtered. Such pre-filtering may be performed in order to extract impurities (e.g., coarse impurities) from the sample before filtering the sample through the filter. As such, the sample preparation system may comprise a pre-filter. The pre-filter may be selected to avoid filtering out bacteria. The pore size of the pre-filter may be at least at least 10 μm, preferably at least 20 μm. The pre-filter preferably has a pore size no greater than 250 μm, more preferably no greater than 150 μm, even more preferably no greater than 80 μm.

The sample preparation system may comprise a chamber in which the sample is collected before filtration. The method may comprise receiving at least 4 ml, at least 5 ml, at least 6 ml, at least 8 ml, at least 10 ml, at least 20 ml, at least 50 ml of sample in the chamber.

The filter may comprise a first side (or upper side) and a second side (or lower side). Filtering the sample through the filter may comprise forcing the sample from the first side to the second side by applying a pressure on the sample at the first side that is above atmospheric pressure. Pressure may be applied by various mechanical means, such as a piston, e.g. a piston moving in the chamber.

Amplifying the lysate preferably comprises extracting the lysate from the filter before amplification. The sample preparation unit may comprise one or more outlets through which the lysate may be transferred from the filter to an amplification unit in which the DNA or RNA amplification is performed. Transferring the lysate from the filter to the amplification unit may comprise establishing a liquid connection between the outlet and an inlet of the amplification unit.

The sample preparation unit may be disposable. The sample preparation unit may be made from a polymeric material.

The amplification unit may perform an isothermal DNA or RNA amplification, e.g. LAMP.

The lysis reagent and/or lysis buffer preferably comprises at least one of sodium hydroxide, sodium dihydrogen phosphate, disodium hydrogen phosphate, Tris, HEPES, and detergent.

More preferably, the lysis buffer comprises at least 250 mM NaOH. The lysis buffer may be provided in a lysis reagent and/or lysis buffer container.

The method may comprise applying a pressure above atmospheric pressure to the lysis reagent and/or buffer when applying it to the filter, e.g. by mechanical actuation of a piston or pouch. The method may comprise flowing the lysis reagent and/or buffer through the filter from the first side to the second side.

The method preferably comprises applying a washing buffer to the filter before applying the lysis reagent/and or buffer to the filter. The washing buffer may comprise purified water. The washing buffer may be provided in a washing buffer container.

The method may comprise applying a pressure above atmospheric pressure to the washing buffer when applying it to the filter, e.g. by mechanical actuation of a piston or pouch. The method may comprise flowing the washing buffer through the filter from the first side to the second side.

The method preferably comprises applying a neutralization reagent and/or neutralization buffer to the filter after applying the lysis reagent and/or buffer to the filter. The method may comprise applying a pressure above atmospheric pressure to the neutralization reagent and/or buffer when applying it to the filter, e.g. by mechanical actuation of a piston or pouch. The method may comprise flowing the neutralization reagent and/or buffer through the filter from the first side to the second side.

The neutralization reagent and/or buffer may comprise a composition for neutralization of the pH value of the sample, preferably wherein the neutralization reagent and/or buffer comprises one or more of Tris, Tris-HCl, and ethylenediaminetetraacetic acid (EDTA). The neutralization reagent and/or buffer may be provided in a neutralization reagent and/or buffer container.

The method preferably comprises applying a rehydration buffer to the filter after applying the neutralization reagent and/or buffer to the filter. The method may comprise applying a pressure above atmospheric pressure to the rehydration buffer when applying it to the filter, e.g. by mechanical actuation of a piston or pouch. The method may comprise flowing the rehydration buffer through the filter from the first side to the second side. Preferably, the rehydration buffer comprises purified water, more preferably wherein the rehydration buffer comprises one or more of KCl, ammonium sulfate, MgSO4, deoxynucleotide triphosphates. Triton X-100 (or a similar detergent) and betaine.

The rehydration buffer may be provided within a rehydration buffer container.

Each of the above-mentioned containers may be configured to rupture and/or release the respective buffer towards the filter when a sufficient force and/or pressure is applied. For example, one or more (e.g., each) of the containers may be configured as a pouch that ruptures upon application of pressure in such manner that the buffer container therein is released into a duct leading to the filter. Alternatively or additionally, one or more needles configured to pierce a respective one of the containers may be provided. In this case, the respective buffer may be released along and/or through said needle.

Amplifying DNA or RNA in the lysate may comprise amplifying DNA or RNA of one or more bacteria, viruses, protists, and/or cells of interest, for example one or more bacteria causing STDs or urinary tract infections.

As an alternative to filtering, centrifugation or evaporation of the sample may be employed in the context to the present disclosure. It has been found, however, that these methods are more difficult to perform in a time efficient manner, in particular, for a test to be performed within a short period of time at home or at a point of care.

In view of the above, the present invention may be considered to provide a concentrated crude lysate comprising the DNA or RNA of one or more pathogens and/or cells of interest. In particular, the invention proposes a method wherein a relatively large sample volume (e.g., at least 5 ml, at least 8 ml, or at least 10 ml) is received in a sample preparation system and the pathogenic cells in this larger volume are concentrated in a filter. In this manner a concentrated lysate may be obtained in a simple and cost-effective manner, even at home or at a point of care. Moreover, it has been found by the inventors that such concentrated lysate may suitably be used in various DNA or RNA amplification techniques, including relatively simple DNA or RNA amplification techniques, such as isothermal amplification and/or LAMP.

### BRIEF DESCRIPTION OF THE DRAWINGS

The subject matter of the invention will be explained in more detail in the following text with reference to preferred exemplary and non-limiting configurations which are illustrated in the attached schematic drawings. These figures are intended for illustrational purposes only. In particular, the disclosure provided by the figures and description is not meant to limit the scope of protection conferred by the invention.

FIG. 1a depicts a perspective drawing of a sample preparation system;

FIG. 1b is a view of the sample preparation system shown from above;

FIG. 1c is a side view of the sample preparation system;

FIG. 1d is a cross-sectional view of the sample preparation system;

FIG. 2a depicts a perspective view of the barrel portion of the sample preparation system;

FIG. 2b illustrates a top view of the barrel;

FIG. 2c is a bottom view of the barrel;

FIG. 2d is a cross-section of the barrel;

FIG. 2e is a side view of the barrel;

FIG. 3a provides a perspective view of the cap;

FIG. 3b shows a side view of the cap;

FIG. 3c shows a cross-section of the cap;

FIG. 3d provides a top view of the cap;

FIG. 3e provides a bottom view of the cap;

FIG. 4a shows a perspective view of the outlet cap;

FIG. 4b is a side perspective of the outlet cap;

FIG. 4c is a top view of the outlet cap;

FIG. 4d is a bottom view of the outlet cap;

FIG. 4e is a cross-section of the outlet cap; and

FIG. 5 is a simplified flow chart of the inventive method.

### DETAILED DESCRIPTION

In the following description the terms “sexually transmitted disease” and “sexually transmitted infection” are used interchangeably to mean an illness which is transmitted through sexual contact between individuals.

While the present invention is described in connection with sexually transmitted infections, a person skilled in the art would understand that the present invention may also be used to test for other types of illnesses including, but not limited to urinary tract infection, urethritis, cystitis, pyelonephritis, gingivitis, periodontitis, oncological conditions (e.g., cancerous or precancerous conditions).

While reference is made herein to DNA amplification, other nucleic acids (e.g., RNA) could be isolated and amplified in accordance with the methods of the present invention.

In the figures provided, one particular system is described which is suitable for carrying out the claimed method. It will be evident to the skilled reader that many adaptations to the described system can be made that will still fulfill the requirements of the inventive method. It is also to be noted that alternative systems could be used for the purposes of the present invention.

The figures provided refer to a sample preparation system 100 which is configured to process, e.g., a urine sample according to the inventive method to provide a lysate usable for home testing for sexually transmitted infections or other diseases. The same system 100 could also be employed for a saliva or mouthwash sample. Known home STD testing kits involve a sampling device where one can provide a swab or small urine sample. This sampling device is then sent off to a lab for processing. In the present invention, however, the sampling, the processing, and the diagnosis can be provided in the privacy of one's home.

A large barrier to home testing is that to reach the desired clinical sensitivity the sample must be properly processed. It may be required, for example, for urine particulates to be filtered out, for bacterial, viral, fungal, cellular, and/or protozoan matter to be isolated from the urine and then processed to prepare the isolated material for testing. While this process is generally performed in a lab, in the present method this sample preparation process can be performed simply and privately by a home user or directly at a point of care. The sample prepared according to the method described herein can then be provided to a corresponding DNA amplification unit for readout of results.

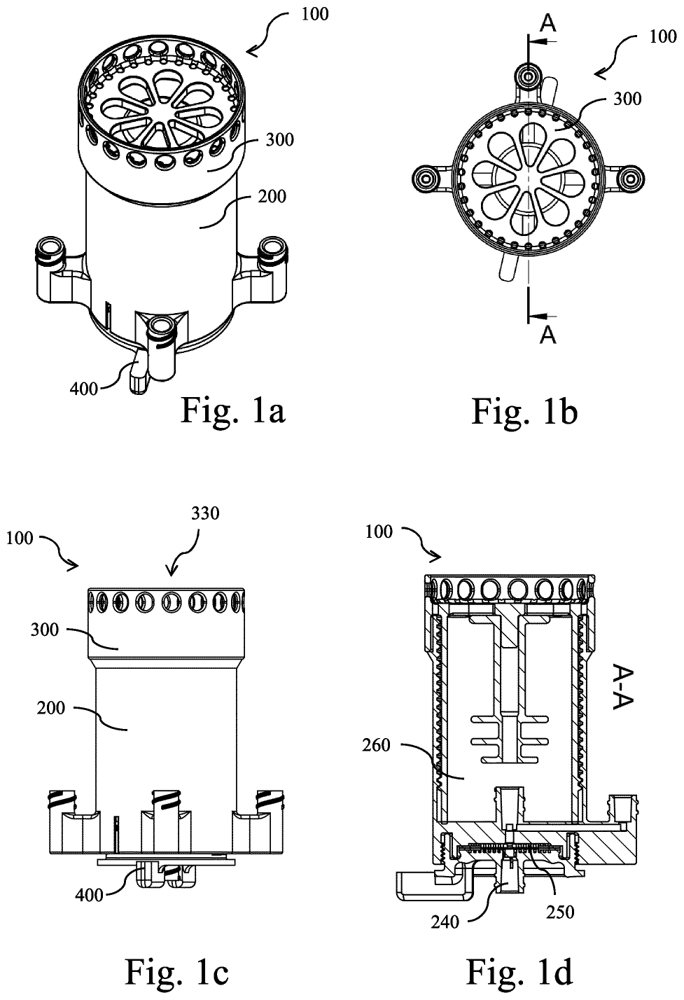

FIG. 1a shows a perspective view of an exemplary sample preparation system 100 which includes a cap 300 coupled to a barrel 200. The sample preparation system 100 comprises an inlet 330 which receives a sample (e.g., a urine sample). The urine is then directed to and collected within a chamber 260 in the barrel 200 of the sample preparation system 100. Once the sample has been collected the sample preparation system is actuated to force the urine sample through a filter 250 which is positioned between the chamber 260 and the outlet 240. The filter 250 collects cellular material and/or bacteria from the urine. Forcing the urine through the filter 250 may also force the urine through the outlet 240. After the urine passes through the filter 250 one or more reagents (not depicted) are actuated so as to introduce specific buffers from the one or more reagents to the collected cellular material on the filter 250. According to the method the one or more reagents include a lysis reagent and/or buffer. The introduction of the buffer/s prepares the cellular material from the urine sample such that it may be used in a separate device (not shown) for PCR amplification and thereby detect a variety of diseases, e.g., sexually transmitted diseases. In the following description each of these components and steps will be described in more detail.

In the sample preparation system depicted in FIG. 1a, the cap 300 and barrel 200 are shown to be cylindrical, however, one or both of the cap 300 and barrel 200 can have other geometries without impeding the functionality of the sample preparation system 100. The sample preparation system 100 is a home use device which is preferably dimensioned to comfortably fit in the hand of a user.

The sample preparation system may be designed to have an upright orientation with the inlet 330 being positioned above the barrel 200 and/or the chamber 260. The sample preparation system 100 is optionally configured to receive between 2 mL and 50 mL of urine, more preferably between 8 and 12 mL of urine, most preferably around 10 mL of urine. Larger urine volumes allow for a larger number of potential bacterial cells to be collected on the filter 250. A minimum volume of 6 ml or 8 mL (e.g., of whole urine) is advantageous in some circumstances in terms of providing highly accurate results. Larger sample volumes allow for a larger number of potential bacterial or other cells or analytes to be collected on the filter 250 which thereby increases the amount of the subsequent DNA or RNA amplification and the accuracy of STI detection. In some implementations diluted urine or other bodily fluid may also be used. Dilution of the provided sample may be advantageous in reducing pressure, for example during the filtering of the sample. The sample may then be diluted to a ratio of anywhere between 1:1 to 1:50, i.e. between undiluted and one part in fifty. The sample may be diluted to a ratio of 1:2, 1:5, 1:10, 1:20 or 1:50. The sample may be diluted with water.

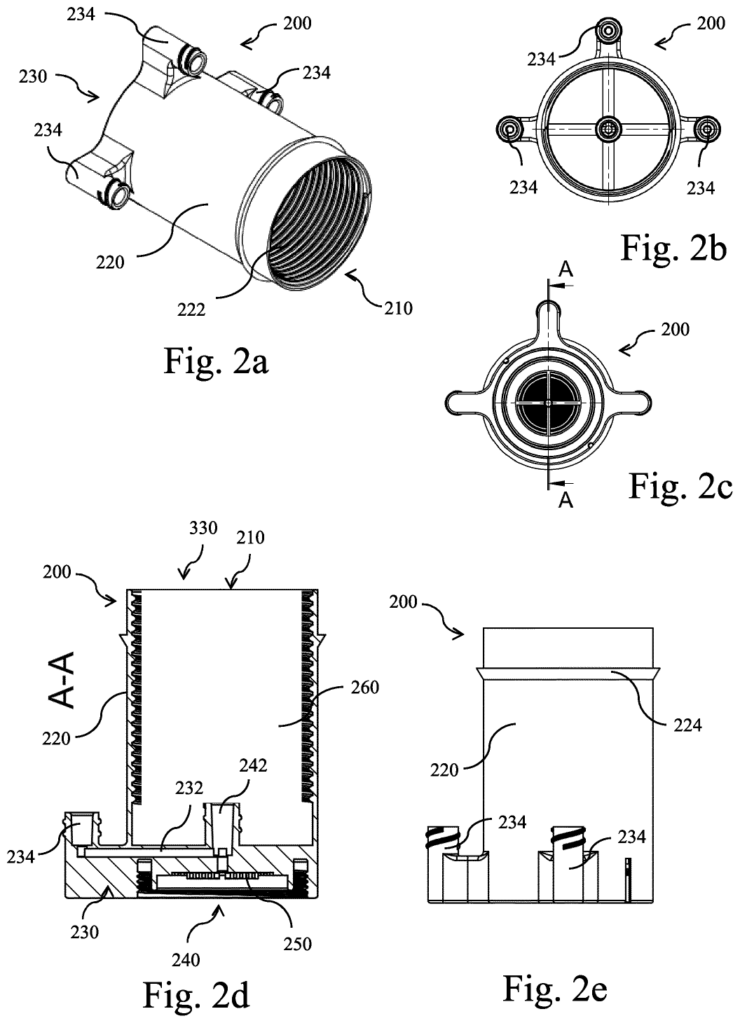

The barrel 200 of one embodiment of the sample preparation system 100 is depicted in FIGS. 2a to 2e having an open side 210 and a base 230. Generally speaking, the barrel 200 may be any shape such that it can accommodate a urine sample. In preferred configurations of the sample preparation system 100 the barrel 200 has a cylindrical wall 220 extending between the open side 210 and the base 230. Optionally, the open side 210 of the barrel 200 may serve as the inlet 330 for receiving a urine sample.

The base 230 of the barrel 200 comprises an outlet conduit 242 extending from the chamber 260 to an outlet 240 for the urine sample. As best viewed in FIG. 2d, a filter 250 is positioned between the chamber 260 and the outlet 240. The filter 250 generally has the smallest pore size sufficient to filter 250 out any cells within the urine sample, specifically cells of bacterial size which may mean a pore size of maximally 1.0 μm, 0.8 μm, 0.6 μm, 0.5 μm, 0.45 μm, 0.4 μm, or 0.22 μm.

The filter 250 may take the form of a membrane or multiple membranes. Where the outlet 240 comprises multiple membranes, including a first membrane closest to the chamber 260 may have a pore size of maximally 250 μm, 150 μm, or 80 μm. A second membrane may have a pore size of maximally 1.0 μm, 0.8 μm, 0.6 μm, 0.5 μm, 0.45 μm, 0.4 μm or 0.22 μm. And potentially a third membrane furthest from the chamber 260 may be included having a pore size of maximally 250 μm, 150 μm, or 80 μm. Gaskets, particularly silicon gaskets, may be positioned between the membranes and/or above and below the filter 250 in order to hold the filter 250 and the individual membranes in place. The membranes are preferably made out of cellulose acetate (CA), mixed cellulose esters (MCE), polycarbonate track etched (PCTE), and/or cellulose nitrate (CN). Alternatively, the membranes may be made from materials such as mixed cellulose esters (MCE), regenerate cellulose (RC), polytetrafluoroethylene polymer (PTFE), Nylon, cellulose mixed esters (CME), polyvinylidene difluoride (PVDF) and/or polyethersulfone (PES). A membrane having a 0.45 μm pore size and formed from or comprising mixed cellulose esters (MCE) is a preferred embodiment. A membrane having a 0.45 μm pore size and formed from or comprising cellulose acetate (CA) is a preferred embodiment. A membrane having a 0.65 μm pore size or smaller is a preferred embodiment.

When the barrel 200 is used together with a cap 300, the inside of the cylindrical wall 220 may further comprise threads such that it can be screwed together with a compatible cap 300. Further the cylindrical wall 220 may comprise a stop feature, e.g. a stop ridge 224, which limits the advancement of the cap 300 (e.g., on the inside and/or on the outside). It will be appreciated by the skilled reader that the threads could alternatively be provided on the inside of the cylindrical wall. Furthermore, it will be appreciated by the skilled reader that any other mechanism allowing for a relative movement between the barrel 200 and the cap 300 could be employed.

One particular example of the filter 250 may be constructed from a silicon plate of 40 Shore being cut in 20 mm inner diameter, 25 mm outer diameter format at 1 mm and 0.5 mm thickness values to create silicon gaskets 354. The 1 mm-thick silicon gasket may be glued within the outlet 240 of the barrel 200 by using a silicon glue. An 80 μm pore size nylon net filter 250 may be placed over the opening of the barrel 200, just above the 1 mm-thick silicon gasket. A 0.5 mm-thick silicon gasket may be placed on top of the nylon net filter 250. A 0.45 μm pore size cellulose acetate (CA) filter 250 may be placed on the silicon gasket. Another 0.5 mm-thick silicon gasket may be placed on the CA filter 250 and finally a 140 μm pore size nylon net filter 250 may be placed.

The presence of the filter 250 positioned between the chamber 260 and the outlet 240 allows for bacterial cells to be retained on the filter 250 before passing through the outlet 240. In a healthy individual urine will contain very few or no bacterial cells. However, a person experiencing an infection, e.g. a sexually transmitted infection, may have bacterial growth within the urethra or bladder which is expelled during urination. The pore size of the filter/membranes can thus be optimized to catch bacterial cells, and optionally to further filter out larger material to keep the bacterial retention membrane from becoming obstructed.

The barrel 200 comprises a chamber 260 for receiving the urine sample. The sample preparation unit may comprise a reception configuration and a preparation configuration. In the reception configuration urine may be introduced into the chamber 260 through the inlet 330 of either the barrel 200 or through a cap 300. After the urine sample is collected in the chamber 260 the sample preparation system 100 may be transitioned into a preparation configuration. Movement from the reception configuration into the preparation configuration may involve compression of the urine in the chamber 260 and/or compression of the chamber 260 itself. This may be accomplished in any number of ways, i.e. using valves and/or pumps, such that a pressure is exerted on the liquid within the chamber 260 and that the outlet 240 is provided as the only means of egress for the liquid sample.

In some embodiments the sample preparation system 100 further comprises a receptacle in fluid communication with the outlet 240, so as to receive and store the filtered liquid sample therefrom.

In some examples which employ a cap 300, transition between the reception configuration and the preparation configuration involves moving the cap 300 to compress the chamber 260. For example, the cap 300 and the barrel 200 may have corresponding threads which join them, such that in the reception configuration the cap 300 is partially screwed onto the barrel 200 and in the preparation configuration the cap 300 is fully screwed onto the barrel 200. By screwing the cap 300 further onto the barrel 200, the size of the chamber 260 is reduced and the urine is forced through the outlet 240. For example, the cap 300 may comprise a piston 350. This piston 350 can further aid in forcing the urine through the outlet 240. Moreover, optional gaskets 354 extending radially from the piston 350 can aid in sealing the chamber 260 such that the urine may only flow through the outlet 240. It is noted that throughout the figures the piston 350 is shown without a sealing gasket provided thereon. Such sealing gasket may be connected to the end of the piston protruding towards the bottom of the chamber. The gasket may close the gap between the piston 350 and the inner peripheral wall of the chamber 260 that is visible in FIGS. 1a and 2c.

In some configurations, the inlet 330 is located on the opposite side of the filter 250 from the outlet 240. This configuration provides for more direct throughput such that excess liquid sample which is not retained on the filter 250 can then be directly disposed of, for example, into the toilet.

The pressure exerted on the liquid within the chamber 260 may depend on the speed with which the sample preparation unit is converted from the reception configuration to the preparation configuration by, for example, screwing the cap 300 onto the barrel 200 using threads. The pressure exerted on the liquid within the chamber 260 may also depend on the type of filter used, wherein filters having smaller pore sizes may lead to higher pressures required to convert the sample preparation unit from the reception configuration to the preparation configuration.

The pressure exerted on the liquid may be greater than atmospheric pressure. The pressure may be at least 1.5 bar (150 kPa), at least 1.7 bar (170 kPa). The pressure exerted on the liquid is preferably not greater than 6.0 bar (600 kPa), more preferably no greater than 4.0 bar (400 kPa).

The sample preparation unit may further comprise one or more fluid conduits 232, as can be seen in FIG. 2d. The one or more fluid conduits may provide a connection to the one or more reagents which are used for processing the cellular material caught in the filter 250. Each fluid conduit may begin adjacent the filter 250 and lead to a port 234 which serves as the connection point to the one or more reagents. One fluid conduit 232 for each buffer may be provided. Alternatively, a plurality of buffers may be supplied through a common conduit 232.

The sample preparation unit may be supplied with the one or more buffers, in particular with one or more buffer containers (preferably, all buffer containers), pre-attached and/or fixedly connected to a respective one of the one or more fluid conduits 232 (not shown in the drawings). This may avoid assembly errors by the user.

The one or more fluid conduits 232 may extend through the barrel 200, for example through the base 230 of the barrel 200.

The ports 234 may be adjacent the cylindrical wall 220 of the barrel 200.

The one or more reagents comprise at least a lysis reagent, but may also comprise a washing buffer, a neutralization reagent and a rehydration buffer. The reagents may be provided as a single unit cartridge which may be connected with the single fluid conduit 232 on the barrel 200. Alternatively, the reagents may comprise multiple containers such as syringes, blisters, pouches, etc. which can then individually be attached to multiple fluid conduits 232. In some cases, at least one actuator is provided which initiates the transfer of the buffers from the reagents to the filter 250. In the case of individual syringes, for example, each syringe has a plunger which can be individually actuated to force the buffer through the fluid conduit to the filter 250. Whereas in the case of a reagent blisters or pouches, each blister may be actuated by a user simply compressing the blister(s) and forcing the reagents through the one or more fluid conduits 232 to the filter 250. As such, each respective blister or pouch may be considered to form an actuator. Alternatively, when a single unit cartridge is provided, an actuator button may initiate the process and in conjunction with a microcontroller provide each buffer in a controlled and timed manner.

The lysis buffer comprises at least one of sodium hydroxide, sodium dihydrogen phosphate, disodium hydrogen phosphate, Tris, HEPES, and detergent. In a preferred embodiment the lysis buffer comprises at least 250 mM of NaOH. The lysis buffer disrupts the cellular membrane leaving the DNA content of any cells present accessible for replication in the form of a lysate.

When present the washing buffer may comprise purified water and or an aqueous solution. The washing buffer may be plain water.

If present, the neutralization reagent and/or buffer is configured to lower and/or neutralize the pH value of the sample. The neutralization reagent and/or buffer may comprise Tris and/or Tris-HCl. A rehydration buffer comprises one or more of KCl, ammonium sulfate, MgSO4, deoxynucleotide triphosphates, detergent, Triton X-100, and betaine. Depending on the specific configuration of the sample preparation system 100 any two or more of the washing buffer, lysis buffer, neutralization buffer and rehydration buffer may be combined into a single buffer for delivery to the filter 250. In some embodiments of the present invention a neutralization buffer may be unnecessary or alternatively one reagent mixture may function as a washing buffer, neutralization buffer, and/or rehydration buffer.

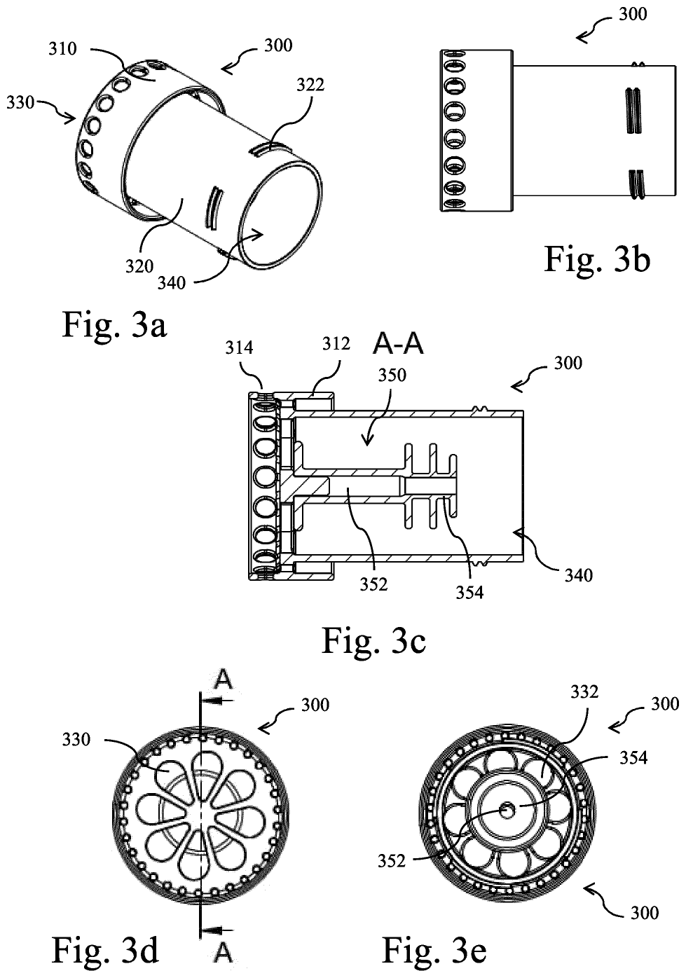

In FIGS. 3a to 3e an exemplary cap 300 is schematically depicted. FIG. 3a shows a perspective view of the cap 300 depicted in FIG. 1a. The cap 300 in some embodiments may have a cylindrical form wherein the cylinder comprises a tube 320 with an open end 340 which may be inserted into the barrel 200. The cap 300 may further comprise a top portion 310 which forms a wall or barrier opposite of the open end 340 of the cap 300. The top portion 310 may have a larger diameter than that of the tube 320. From the outer circumference of the top portion 310 a lip 312 may extend downwards toward the open end 340 of the cylinder. The larger dimension of the top portion 310 and/or the lip 312 can make it easier for the user to grip the cap 300 and then push or twist it down into the barrel 200.

Further, the cap 300 may have an inlet 330 at the top for receiving the urine sample. The inlet 330 may comprise one or more holes in the cap 300 through which urine can pass. In the example provided in FIG. 2d the inlet 330 comprises eight petal-shaped holes which are oriented in a radially symmetric pattern around the central axis of the cap 300. This configuration may help to guide urine down into the barrel 200. It will be appreciated, however, that many other designs are possible in this regard. Alternatively or additionally, the cap could be provided with an upper funnel (not shown in the figures), which makes it easier to catch the sample (e.g., when whole urine is employed).

Inside the barrel 200, there may be provided an internal funnel-shaped portion having a wider section and a narrower section. The wider section may serve for guiding urine into the narrower section. The narrower section may be configured to seal with a piston 350 when the piston 350 is moved through the chamber 260.

From the top portion 310 of the cap 300 a wall may extend from the periphery upwards to create a cup 314 formation. The cup 314 or an upper funnel formation makes it easier to collect the urine, prevent splashing and guide the urine into the barrel 200. In some configurations the cup 314 may include a number of holes extending radially outwards through the wall.

In some particularly advantageous configurations, the cap 300 also comprises a piston 350, as shown in FIG. 3c. The piston 350 can be affixed on the lower face of the top portion 310 and extend within the tube 320 toward the open end 340 of the cap 300. The piston 350 can have an elongated portion 352 which is attached to the top portion 310 of the cap 300 and may further comprise ribs 354 extending radially outward therefrom. The piston 350 can assist in forcing the urine sample out through the outlet 240 of the barrel 200.

When the piston 350 is employed, the chamber 260 within the barrel 200 may have a smaller diameter than the barrel 200 and corresponding to the diameter of the piston 350 such that the movement of the piston 350 through the chamber 260 blocks the backward flow of the urine.

Along the tube 320 one or more threads may extend circumferentially outward. These threads are configured to mate with corresponding threads on the inside of the barrel 200 so that the cap 300 and the barrel 200 can be screwed together.

Additionally, the cap 300 may comprise a pre-filter 332 positioned such that it can filter urine that passes from the inlet 330 into the chamber 260. In this way larger particulates can be prevented from entering the chamber 260 for the urine sample. The pre-filter 332 may also comprise multiple membranes having different pore sizes. In some cases the pore sizes of the pre-filter 332 may be no greater than 250 μm, 150 μm, or 80 μm. Alternatively, the pore sizes of the pre-filter 250 may be no less than 0.6 μm, 0.8 μm, 1.0 μm, 10 μm, or 20 μm. The pore sizes of the pre-filter may be 10 μm, 20 μm, 30 μm, 40 μm, 80 μm, 100 μm, 120 μm, 140 μm, 160 μm, 180 μm, 200 μm or in any range therebetween. In some cases the pre-filter 332 is positioned within the inlet 330 of the cap 300.

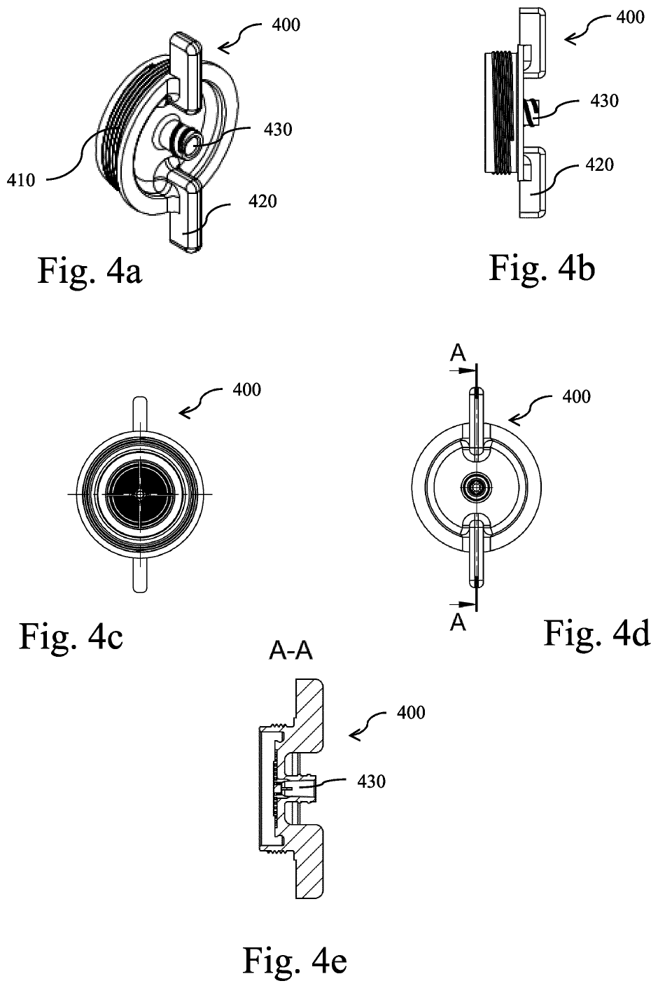

As previously discussed, one or more filters 250 are positioned in the barrel 200, these filters 250 may be held in place by an outlet cap 400 which is depicted in FIGS. 4a to 4e. The outlet cap 400 may have a threaded portion 410 for screwing into a corresponding thread located at the outlet 240 of the barrel 200. Other connections may be used as well.

Tabs 420 may be provided on the outlet cap 400 to aid in screwing the outlet cap 400 into the outlet 240. The outlet cap 400 may comprise a dispensing port 430 which connects to the outlet 240 of the barrel 200, together forming a channel for dispensing the prepared sample. The dispensing port 430 may take the form a Luer-lock or similar liquid-tight connection for secure docking of the sample preparation system 100 to a DNA amplification unit. Alternatively, the barrel 200 itself may comprise at the outlet 240 or at some other location a docking port for connection to the DNA amplification unit.

The outlet cap 400 may enable the positioning and replacement of the filter 250 and any gaskets present and may enable easier production of the sample preparation system 100.

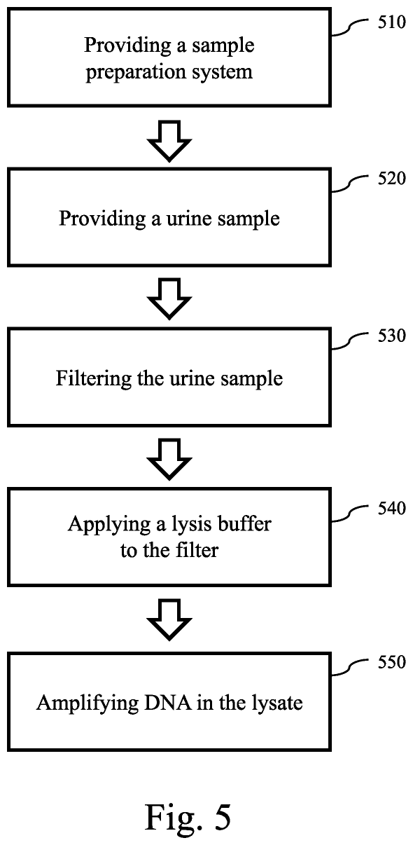

FIG. 5 illustrates a flow chart of the method according to the present invention. The method comprises the step 510 of providing a sample preparation system 100. The sample preparation system 100 includes a filter 250 having an appropriate pore size as described above.

Step 520 of the method involves providing a whole urine sample to the sample preparation system 100 and collecting the urine in the sample preparation system 100. The urine sample should ideally be first catch urine as this likely contains a higher concentration of any analyte of interest present. As previously discussed, a larger urine volume can lead to higher concentrations of cellular material which is retained in the filter 250. Thus, larger volumes of urine, e.g. 8 ml of urine, 10 ml of urine or greater are advantageous and provide more reliable results. Then in step 530 the sample preparation system 100 is actuated by the user to force the urine through the filter 250. This actuation may involve any number of mechanisms suitable for increasing the pressure within the chamber 260 such that the urine is forced through the filter 250. This can involve two parts of the sample preparation unit 100 sliding, twisting or collapsing toward one another such that a pressure greater than atmospheric pressure is placed on the urine sample therein.

Following filtering of the sample, in step 540 a lysis buffer is delivered to the cellular material provided on the filter 250. This step may involve pressing one or more buttons, compressing blisters, turning switches, etc. such that the lysis buffer is delivered to the filter. The lysis buffer disrupts the membranes of any captured cells, such that the DNA within the cells is made available in the form of a lysate.

The step 540 of applying the lysis buffer may additionally involve applying a washing buffer, a neutralization buffer, and/or a rehydration buffer to the filter.

A washing buffer is formulated to rinse away remaining fluid urine components and/or to small molecules (e.g., urobilin) that may act as an amplification inhibitor. The neutralization buffer is formulated to neutralize the pH of the remaining fluid. These buffers allow the cells remaining in the filter 250 to be lysed without suffering negative effects from any remaining urine, which can contain many bodily waste products and be somewhat acidic. The lysis buffer is provided to the filter 250 to disrupt the cellular membranes of the bacteria, rendering the DNA contents of the cells available for binding. The rehydration buffer may be used if DNA amplification factors are provided within a lyophilized pellet which require rehydration before they become activated.

The provision of the lysis buffer to the cellular material gathered on the filter 250 prepares the sample for introduction into a compatible analysis unit which can test the DNA sample for a variety of sexually transmitted (or other) diseases. In some cases it may be advantageous to connect the sample preparation system 100 to the analysis unit before or during step 540, particularly when the lysis buffer is provided to the cell sample.

After the lysate has been prepared, in step 550 the lysate may be extracted through an outlet 240. This extraction may be done directly into the DNA amplification unit or collected in an intermediate step before amplification. In some implementations of the method step 540 and step 550 may partially overlap in that one or more of the buffers are provided in order to promote or to fully provide for extraction of the lysate and initiate the beginning of DNA amplification. Amplification of the DNA according to step 550 may be performed using traditional PCR involving thermocycling or advantageously using isothermal DNA amplification methods such as Loop-Mediated Isothermal Amplification (LAMP). Without wanting to be bound by theory, it is believed that isothermal amplification or LAMP may be particularly suitable for amplifying crude lysate. This is, because Taq and Pfu polymerases are believed to be more sensitive to amplification inhibitors and at least some other PCR method may require a more sophisticated DNA extraction.

In order to promote ease of home use of the sample preparation system 100, instructions for preparing the sample may be provided in a software application running on a mobile telephone or a computer. Such instructions may include the steps of actuation of the sample preparation system 100 including any incubation periods or wait times and the order of actuation of the buffers.

While the invention has been illustrated and described in detail in the drawings and foregoing description, such illustration and description are to be considered illustrative or exemplary and non-restrictive; the invention is thus not limited to the disclosed embodiments. Variations to the disclosed embodiments can be understood and effected by those skilled in the art and practicing the claimed invention, from a study of the drawings, the disclosure, and the appended claims. In the claims, the word “comprising” does not exclude other elements or steps, and the indefinite article “a” or “an” does not exclude a plurality and may mean “at least one”.

1. A method for sample preparation and DNA or RNA amplification from a urine sample, a saliva sample and/or a mouthwash sample by a user, wherein the method comprises the steps of
(i) providing a sample preparation system comprising a filter,
(ii) providing a urine sample, a saliva sample and/or a mouthwash sample,
(iii) filtering at least a portion of the sample through the filter by applying a pressure on the sample that is above atmospheric pressure, thereby retaining bacteria, fungi, viruses, protists and/or cancerous or precancerous cells in the filter,
(iv) applying a lysis reagent to the filter,
(v) amplifying DNA or RNA of the bacteria, fungi, viruses, protists and/or cancerous or precancerous cells contained in the lysate,
wherein the user performs at least steps (ii) to (v).

2. The method of claim 1, further comprising the step of
(vi) discarding the urine, saliva or mouthwash that has passed through the filter.

3. The method of claim 1 or 2, wherein the urine sample is whole urine, preferably wherein the whole urine is micturated 1 hour or less before filtering the urine in step (iii).

4. The method of any one of the previous claims, wherein the sample preparation system comprises a pre-filter and wherein the sample passes through the pre-filter before passing through the filter.

5. The method of any one of the previous claims, wherein amplifying DNA or RNA in the lysate may comprise amplifying DNA or RNA of one or more bacteria, fungi, protists, or viruses, preferably one or more bacteria or protists causing STDs or urinary tract infections.

6. The method of any one of the previous claims, wherein amplifying DNA in the lysate comprises extracting the lysate from the filter before amplification, preferably wherein the sample preparation unit comprises one or more outlets through which the lysate may be transferred from the filter to an amplification unit in which the DNA amplification is performed.

7. The method of claim 6, further including the step of establishing a liquid connection between the one or more outlets of the sample preparation unit and an inlet of the amplification unit.

8. The method of any one of the previous claims, wherein the filter comprises a first side and a second side, wherein filtering the sample through the filter comprises forcing the sample from the first side to the second side by applying a pressure on the sample at the first side that is above atmospheric pressure, preferably wherein the pressure is applied by a mechanical means, more preferably wherein the mechanical means involves moving a piston within a chamber of the sample preparation system.

9. The method of any one of the previous claims, wherein step (iii) comprises passing at least 5 ml, at least 10 ml, at least 20 ml, or at least 50 ml of the sample through the filter.

10. The method of any one of the previous claims, wherein step (iv) further comprises applying a pressure above atmospheric pressure to the lysate buffer when applying it to the filter, preferably wherein the pressure is provided by mechanical actuation of a piston or pouch.

11. The method of any one of the previous claims, wherein the filter has a pore size no greater than 1.0 μm, preferably no greater than 0.8 μm, more preferably no greater than 0.6 μm, even more preferably no greater than 0.5 μm, most preferably no greater than 0.45 μm; alternatively the filter has a pore size no greater than 0.4 μm, and more preferably no greater than 0.22 μm.

12. The method of any one of the previous claims, wherein step (iv) further comprises applying a washing buffer to the filter, preferably wherein the washing buffer comprises purified water.

13. The method of any one of the previous claims, wherein the lysis buffer comprises at least one of sodium hydroxide, sodium dihydrogen phosphate, disodium hydrogen phosphate, Tris, HEPES, and detergent, more preferably wherein the lysis buffer comprises at least 250 mM NaOH.

14. The method of any one of the previous claims, wherein step (iv) further comprises applying a neutralization reagent to the filter, wherein the neutralization reagent comprises a composition for neutralization of the pH value of the sample, preferably wherein the neutralization reagent comprises an alkaline reagent and/or wherein the neutralization buffer comprises one or more of Tris, Tris-HCl, and ethylenediaminetetraacetic acid (EDTA).

15. The method of any one of the previous claims, wherein step (iv) further comprises applying a rehydration buffer to the filter, preferably wherein the rehydration buffer comprises purified water, more preferably wherein the rehydration buffer comprises water having one or more of KCl, ammonium sulfate, MgSO4, and deoxynucleotide triphosphates, detergent, Triton X-100, and betaine.

16. The method of any one of the previous claims, wherein step (v) comprises amplifying DNA by Loop-Mediated Isothermal Amplification (LAMP).
